(19) United States
(12) Patent Application Publication
(10) Pub. No.: US 2006/0177438 A1
(43) Pub. Date: Aug. 10, 2006

(54) Methods of altering absorption of hydrophobic compounds

(75) Inventors: Alan S. Kopin, Wellesley, MA (US); Martin Carey, Wellesley, MA (US); David Wang, Newton, MA (US)

Correspondence Address: PALMER & DODGE, LLP;KATHLEEN M. WILLIAMS, 111 HUNTINGTON AVENUE, BOSTON, MA, 02199, US

(73) Assignee: New England Medical Center

(21) Appl. No.: 11/053,553

(22) Filed: Feb. 8, 2005

(51) Int. Cl. A61K 39/395; A61K 31/5513

(52) U.S. Cl. 424/143.1; 514/221

(57) ABSTRACT

This invention presents methods of increasing intestinal motility rates in order to decrease intestinal absorption of cholesterol. Furthermore, this invention presents methods of modulating intestinal motility in order to influence positively the amount of drug or nutrient absorption from the intestine, especially of hydrophobic drugs or nutrients. The instant methods comprise modulating the rate of intestinal motility through the use of agonists and/or antagonists of the cholecystokinin-1 receptor.

### GOVERNMENT INTEREST

This invention was made with government support under grants DK54012, DK36588, DK34854, DK52911, DK46767, DK 34928 from the National Institutes of Health (US Public Health Service). The Government has certain rights in this invention.

### FIELD OF THE INVENTION

This invention relates to methods encompassing the cholecystokinin-1 receptor (CCK-1 R) for altering the absorption of hydrophobic compounds and for treating diseases including hypercholesterolemia.

### BACKGROUND

Cholecystokinin (CCK) is a neuropeptide hormone secreted from gut endocrine cells which plays a significant role in many physiological processes including regulation of satiety, bowel motility, gastric emptying, insulin secretion, pancreatic enzyme secretion and neurotransmission. CCK is responsible indirectly for stimulating the digestion and absorption of fat, carbohydrate and protein. CCK is secreted mostly by the duodenum, the first segment of the small intestine, and causes the release of digestive enzymes and bile from the pancreas and gallbladder, respectively. It also acts as a hunger suppressant.

CCK is derived from the 115 amino acid peptide which has a sequence of: mnsgvclcvl mavlaagalt qpvppadpag sglqraeeap rrqlrvsqrt dgesrahlga llaryiqqar kapsgrmsiv knlqnldpsh risdrdymgw mdfgrrsaee yeyps, (SEQ ID NO:1, Accession number AAA53094). CCK exists in multiple molecular forms in the circulation, including forms that are 58, 39, 33, 22, 8 and 4 amino acids in length. Following ingestion of dietary fat, carbohydrate and protein, a variety of CCK molecular forms are secreted from endocrine mucosal I cells that are located mainly in the duodenum and proximal jejunum.

CCK-33 was the original form purified from porcine intestine. The polypeptide hormone, CCK-33, has the amino acid sequence: Lys-Ala-Pro-Ser-Gly-Arg-Val-Ser-Met-Ile-Lys-Asn-Leu-Gln-Ser-Leu-Asp-Pro-Ser-His-Arg-Ile-Ser-Asp-Arg-Asp-Tyr(SO₃H)-Met-Gly-Trp-Met-Asp-Phe-NH₂ (SEQ ID NO:2). Cholecystokinin 58 consists of amino acids 46 to 103 of SEQ ID NO:1; cholecystokinin 39 consists of amino acids 65-103 of SEQ ID NO:1; cholecystokinin 33 consists of amino acids 71-103 of SEQ ID NO:1; cholecystokinin 12 consists of amino acids 96-103 of SEQ ID NO:1, and cholecystokinin 8 consists of amino acids 96-103 of SEQ ID NO:1,

The C-terminal octapeptide CCK-8 is well conserved between species and is the smallest form that retains the full range of biological activities.

There are two different subtypes of CCK receptors: CCK 1 and CCK 2. They are ˜50% homologous. The CCK 1 receptor binds mostly to CCK. The CCK 1 receptors are located in organs such as the brain, pancreas, gallbladder, small intestine and colon, and exhibit high affinity for CCK-8s and a lower affinity for the corresponding desulphated fragment, CCK-8d, for CCK-4, and gastrin.

The CCK 2 receptor (CCK-2R) is found in the brain, on smooth muscle cells, and on parietal cells of the stomach (also known as the “gastrin” receptor). Binding studies on brain membranes and parietal cells comparing the relative affinities for agonists show a 6-10 fold and a 1-2 fold higher affinity for CCK than for gastrin, respectively (Jensen, R. T. et al., in Gastrointestinal Endocrinology: Receptors and Post-Receptor Mechanisms, Harcourt Brace Jovanovich, San Diego, p. 95). CCK-2R displays a high affinity for the sulphated octapeptide fragment (CCK-8s), the desulphated octapeptide (CCK-8d), gastrin, CCK-4 (the C-terminal tetrapeptide of CCK), and pentagastrin (CCK-5), and resemble gastrin receptors in their agonist selectivity. CCK 2 receptors may play a role in anxiety, modulation of pain, memory, satiety, and panic disorders. CCK-2R is the most abundant receptor subtype in the brain and stomach.

The amino acid sequence of human CCK-1R is:

mdvvdsllvn gsnitppcel glenetlfcl dqprpskewq
pavqillysl ifllsvlgnt lvitvlirnk rmrtvtnifl
lslavsdlml clfcmpfnli pnllkdfifg savcktttyf
mgtsvsvstf nlvaislery gaickplqsr vwqtkshalk
viaatwclsf timtpypiys nlvpftknnn qtanmcrfll
pndvmqqswh tflllilfli pgivmmvayg lislelyqgi
kfeasqkksa kerkpsttss gkyedsdgcy lqktrpprkl
elrqlstgss sranrirsns saanlmakkr virmlivivv
lfflcwmpif sanawraydt asaerrlsgt pisfilllsy
tsscvnpiiy cfmnkrfrlg fmatfpccpn pgppgargev
geeeeggttg aslsrfsysh msasvppq

(SEQ ID NO:3, Accession No. AAP84362)

The amino acid sequence of human CCK-2R is:

mellklnrsv qgtgpgpgas lcrpgaplln sssvgnlsce
pprirgagtr elelairitl yaviflmsvg gnmliivvlg
lsrrlrtvtn afllslavsd lllavacmpf tllpnlmgtf
ifgtvickav sylmgvsvsv stlslvaial erysaicrpl
qarvwqtrsh aarvivatwl lsgllmvpyp vytvvqpvgp
rvlqcvhrwp sarvrqtwsv llllllffip gvvmavaygl
isrelylglr fdgdsdsdsq srvrnqgglp gavhqngrcr
petgavgeds dgcyvqlprs rpaleltalt apgpgsgsrp
tqakllakkr vvrmllvivv lfflcwlpvy santwrafdg
pgahralsga pisfihllsy asacvnplvy cfmhrrfrqa
cletcarccp rpprarpral pdedpptpsi aslsrlsytt
istlgpg

(SEQ ID NO:4, Accession No.NP_{—}795344 NP_{—}000722)

It has been estimated that up to half of all deaths in the United States are caused by complications of atherosclerosis. Atherosclerosis is a disease in which cholesterol and its fatty acid esters accumulate in the wall of arteries, forms bulky plaques that inhibit the flow of blood, forms a clot, obstructing an artery and causes a heart attack or stroke. Hypercholesterolemia is one of the major risk factors for the development of atherosclerotic disease. Hypercholesterolemia has been suggested to contribute to atherosclerosis by: (1) chemical injury to endothelial cells lining the intima of arteries; (2) stimulating adherence of monocytes and macrophages to the site of injury; and (3) providing increased lipid substrates for uptake by monocytes and arterial smooth muscle cells.

The risk of death from coronary artery disease has a continuous and graded relation to total serum cholesterol levels greater than 180 mg/dL. The cholesterol for the atherosclerotic plaque is derived from particles called low-density lipoprotein (LDL) that circulate in the bloodstream. Moreover, the acceleration of atherosclerosis is positively correlated with elevations of LDL cholesterol. In general, the more LDL there is in the blood, the more rapidly atherosclerosis develops. In contrast, elevation of high-density lipoprotein cholesterol HDL cholesterol has a negative correlation with atherosclerosis. Therefore, treatment regimes traditionally have been designed to reduce LDL cholesterol and/or elevate HDL cholesterol. These treatments include dietary intervention, exercise, and pharmacotherapy. Because many patients have difficulty achieving and maintaining a low-fat diet and a regular exercise program, and because these interventions are not a panacea, drug therapy is widespread.

Epidemiological, clinical, genetic, experimental, and pathological studies have clearly established the primary role of lipoproteins in atherogenesis (i.e., the formation of atherosclerotic plaques). Lowering plasma cholesterol concentrations reduces the availability of atherogenic lipoproteins and presumably, the accumulation of cholesterol in the intima of arteries. Efforts to lower plasma cholesterol have become fundamental to the practice of preventative cardiology, and their use in both healthy patients and those who already have coronary disease has materially contributed to the 50% reduction observed in mortality from coronary heart disease in the United States over the past two decades (Havel, et al, Management of Primary Hyperlipidemia, The New England Journal of Medicine, 332 (22):1491-1498 (1995)). The claimed invention encompasses methods of lowering plasma cholesterol levels in patients diagnosed with hypercholesterolemia.

The handling of cholesterol by the intestine involves a balance between absorption, excretion and metabolism by gut microflora. Between 34-57% of dietary cholesterol is absorbed by the human intestine. The efficiency of cholesterol absorption is affected by the intestinal transit time. (Lichenstein A. H. Ann. Med. 22(1):49-52 (1990)).

### SUMMARY OF THE INVENTION

This invention presents methods of increasing the small intestinal motility rates in order to decrease the small intestinal absorption of cholesterol by decreasing the small intestinal transit time of cholesterol. Furthermore, this invention presents methods of modulating small intestinal motility rates in order to modulate the amount of small intestinal absorption of hydrophobic compounds such as drugs or nutrients. The instant methods comprise modulating the rate of small intestinal motility through the use of agonists and/or antagonists of the cholecystokinin-1 (CCK-1) receptor.

This invention encompasses a method of reducing the level of cholesterol in an individual, comprising administering to said individual an agonist of a cholecystokinin-1 receptor (CCK-1R), whereby the level of cholesterol is reduced in said individual. In an aspect of this method, the individual is a human. In another aspect of this method, the CCK-1R is a human CCK-1R. In an aspect of this method of reducing the level of cholesterol in an individual, the agonist can include, but is not limited to the following; CCK or a fragment, analog or derivative thereof, GI5269, GI0122, GW5823, GW7854, GW7178, GW8573, a 1,4-benzodiazepine, a 1,5-benzodiazepine, PD170292, SR-146,131, and a CCK-1R specific agonistic antibody or antigen binding fragment thereof.

This invention encompasses a method of reducing the level of cholesterol in an individual, comprising administering to said individual an agonist of a cholecystokinin-1 receptor (CCK-1R). The type of cholesterol reduced is selected from the group consisting of total serum cholesterol, low-density lipoprotein (LDL), very low-density lipoprotein (VLDL), VLDL+LDL, chylomicron cholesterol, cholesterol esters, and unbound cholesterol. In another aspect of this method, the administration of the CCK-1R agonist reduces the cholesterol level by a statistically significant amount, said statistically significant amount of reduction in cholesterol level being at least 5%, 6%, 7%, 8%, 9%, 10%, 15%, 20%, or more.

This invention encompasses a method of reducing the level of blood cholesterol in an individual, comprising administering to said individual an agonist of a cholecystokinin-1 receptor (CCK-1R). The high-density lipoprotein (HDL) is increased in said individual.

This invention encompasses a method of reducing the level of serum (plasma or blood) cholesterol in an individual, comprising administering to said individual an agonist of a cholecystokinin-1 receptor (CCK-1R), and further comprising measuring the level of cholesterol in said individual either prior to administering said agonist or after administering said agonist; the type of cholesterol measured is selected from the group consisting of total serum cholesterol, low-density lipoprotein (LDL), very low-density lipoprotein (VLDL), VLDL+LDL, and chylomicron cholesterol, cholesterol esters, and unbound cholesterol. In some embodiments the level of serum cholesterol is measured both before and after administering the CCK-1R agonist.

This invention encompasses a method of reducing the level of cholesterol in an individual in which the intestinal motility is increased in said individual. In an aspect of this method, the small intestinal motility is increased in said individual. In a further aspect of this method of reducing the level of cholesterol in an individual, the intestinal motility is measured. The intestinal motility in said individual can be measured either prior to administering said agonist or after administering said agonist. The intestinal motility can be measured by using a radioopaque tracer or microtelemetry. In some embodiments the intestinal motility is measured both before and after administering the CCK-1R agonist.

This invention encompasses a method of reducing the level of cholesterol in an individual, comprising administering to said individual an agonist of a cholecystokinin-1 receptor (CCK-1R), and further comprising administering an additional cholesterol reducing agent to said individual. This cholesterol reducing agent can include, but is not limited to the following: a lipase inhibitor, a bile acid sequestrant such as cholestryamine, cholestipol, or cholesevelan HCl and DEAE-Sephadex (Secholex®, Polidexide®), as well as clofibrate, lipostabil (Rhone-Poulenc), Eisai E-5050 (an N-substituted ethanolamine derivative), imanixil (HOE-402) tetrahydrolipstatin (THL), istigrnastanylphosphorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, neomycin, p-aminosalicylic acid, aspirin, poly-(diallylmethylamine) derivatives such as disclosed in U.S. Pat. No. 4,759,923, quaternary amine poly(diallyldimethylammonium chloride) and ionenes such as disclosed in U. S. Pat. No. 4,027,009, lovastatin, parvastatin, simvastatin, probucol, gemfibrozil, endomycin, niacin, an inhibitor of HMG CoA reductase, synvolin, pravastain, an antihyperlipoproteinemic, an ACAT inhibitor, an HMG CoA synthase inhibitor, a squalene epoxidase inhibitor, and other known serum cholesterol lowering agents which lower cholesterol through a mechanism other than by the inhibition of the enzyme HMG CoA reductase or squalene. In another aspect of this method of reducing the level of cholesterol in an individual, the route of administration includes but is not limited to, the oral, nasal, or parenteral administration of an agonist of a cholecystokinin-1 receptor (CCK-1R).

This invention encompasses a method of treating a condition including, but not limited to, a hypercholesterolemia, atherosclerosis, myocardial infarction, stroke, gallstones, Alzheimer's disease, constipation, gastric stasis, irritable bowel syndrome, and inflammatory bowel disease, comprising administering to an individual in need thereof an agonist of a CCK-1R in a therapeutically effective amount, wherein intestinal motility is increased in said individual. In an aspect of this embodiment, the agonist can include, but is not limited to the following; CCK or a fragment, analog or derivative thereof, GI5269, GI0122, GW5823, GW7854, GW7178, GW8573, a 1,4-benzodiazepine, a 1,5-benzodiazepine, PD170292, SR-146,131, and a CCK-1R specific agonistic antibody or antigen binding fragment thereof. In another aspect of this method of treating a condition, the intestinal motility is increased in said individual. In another aspect of this method, the small intestinal motility is increased in said individual. In a further aspect of this method of treating a condition, the intestinal motility is measured. In another aspect of this method of treating a condition, the intestinal motility in said individual is measured either prior to administering said agonist or after administering said agonist. In yet another rapsect of this method, intestinal motility is measured both before and after administration of a CCK-1R agonist. In another aspect of this method of treating a condition, the intestinal motility can be measured by using a radioopaque tracer or microtelemetry, but is not limited to these means of measurements; equivalent means of measuring the intestinal motility may be substituted. An aspect of this method of treating a condition includes, but is not limited to, the oral, nasal, or parenteral administration of an agonist of a cholecystokinin-1 receptor (CCK-1R).

This invention encompasses also a method of increasing the intestinal absorption of a drug or nutrient, comprising administering to an individual a composition comprising an antagonist of CCK-1R in an amount sufficient to decrease intestinal motility in said individual, whereby the intestinal absorption of said drug or nutrient is increased in said individual. In an aspect of this method, the drug or nutrient is hydrophobic. In an aspect of this method, the hydrophobic drug or nutrient has an octanol/water partition coefficient in the range from about 0.01 to about 25. Additional preferred embodiments of this method comprise administration of a hydrophobic drug or nutrient that has an octanol/water partition coefficient in one or more of the following ranges: about 0.1 to about 25, about 0.5 to about 25, about 1 to about 25, and about 0.5 to about 10. This invention encompasses a method of increasing the intestinal absorption of a drug or nutrient comprising administering to an individual a composition comprising an antagonist of a CCK-1R. The antagonist can include, but is not limited to, tarazepide, devazepide, lintitript, dexioxiglumide, loxiglumide, JMV179, JMV 180, SR-27,897, L-364,718, and a CCK-1R specific antagonistic antibody or antigen binding fragment thereof. In an aspect of this method of increasing the intestinal absorption of a drug or nutrient, the intestinal absorption of said drug or nutrient is increased by a statistically significant amount. In an aspect of this method of increasing the intestinal absorption of a drug or nutrient, the intestinal absorption of said drug or nutrient is increased by 5%, 10%, 15%, 20%, 25%, 30%, 40%, 50% or more. In an aspect of this method of increasing the intestinal absorption of a drug or nutrient, the small intestinal motility of said individual is decreased. In an aspect of this method of increasing the intestinal absorption of a drug or nutrient comprising administering to an individual a composition comprising an antagonist of CCK-1R, the antagonist is administered orally, nasally, or parenterally. In an aspect of this method of increasing the intestinal absorption of a drug or nutrient comprising administering to an individual a composition comprising an antagonist of a CCK-1R, the individual is a human. In an aspect of this method of increasing the intestinal absorption of a drug or nutrient comprising administering to an individual a composition comprising an antagonist of a CCK-1R, the CCK-1R is a human CCK-1R.

This invention encompasses a method of increasing intestinal motility in an individual, comprising administering to said individual an agonist of CCK-1R in an amount sufficient to increase the intestinal motility of said individual. In an aspect of this method of increasing intestinal motility in an individual, comprising administering to said individual an agonist of CCK-1R, the agonist includes, but is not limited to GI5269, GI0122, GW5823, GW7854, GW7178, GW8573, a 1,4-benzodiazepine, a 1,5-benzodiazepine, PD170292, SR-146,131, CCK, or fragment, analog or derivative thereof, and a CCK-1R specific agonist antibody or antigen binding fragment thereof. In an aspect of this method of increasing intestinal motility in an individual, the small intestinal motility is increased. An aspect of this method includes, but is not limited to, the oral, nasal, or parenteral administration of an agonist of a cholecystokinin-1 receptor (CCK-1R). In an aspect of this method of increasing intestinal motility in an individual, the individual is a human. In an aspect of this method, the CCK-1R is a human CCK-1R. Preferably, intestinal motility is increased by a statistically significant amount. More preferably, intestinal motility is increased by 5%, 10%, 15%, 20%, 25%, 30%, 40%, 50% or more.

This invention encompasses a method of decreasing intestinal motility in an individual, comprising administering to said individual an antagonist of CCK-1R in an amount sufficient to decrease intestinal motility of said individual. In an aspect of this method, the antagonist includes, but is not limited to tarazepide, devazepide, lintitript, dexioxiglumide, 1oxiglumide, JMV179, JMV 180, SR-27,897, L-364,718, and a CCK-1R specific antagonist antibody and antigen binding fragment thereof. In an aspect of this method, the small intestinal motility is decreased. An aspect of this method includes, but is not limited to, the oral, nasal, or parenteral administration of an antagonist of a cholecystokinin-1 receptor (CCK-1R). In an aspect of this method, the CCK-1R is a human CCK-1R. In an aspect of this method, the individual is a human. In an aspect of this method, the CCK-1R is a human CCK-1R. Preferably, intestinal motility is increased by a statistically significant amount. More preferably, intestinal motility is decreased by 5%, 10%, 15%, 20%, 25%, 30%, 40%, 50% or more.

This invention encompasses a pharmaceutical composition for reducing the serum cholesterol level of an individual, comprising an agonist of CCK-1R in an amount sufficient to decrease intestinal absorption of cholesterol in said individual, and a pharmaceutically acceptable carrier. In an embodiment of this pharmaceutical composition, the pharmaceutical composition comprises an agonist of CCK-1R in an amount sufficient to decrease intestinal absorption of cholesterol in said individual, a pharmaceutically acceptable carrier, and further comprises a therapeutically effective amount of a cholesterol reducing agent.

This invention encompasses a pharmaceutical composition for increasing the intestinal absorption of a therapeutic drug or nutrient in an individual, comprising an antagonist of CCK-1R in an amount sufficient to increase intestinal absorption of said therapeutic drug or nutrient, and a pharmaceutically acceptable carrier. An embodiment of this method encompasses a pharmaceutical composition for increasing the intestinal absorption of a therapeutic drug or nutrient in an individual, comprising an antagonist of CCK-1R in an amount sufficient to increase intestinal absorption of said therapeutic drug or nutrient, a pharmaceutically acceptable carrier, and further comprises a therapeutically effective amount of said therapeutic drug or nutrient.

An embodiment of the invention encompasses a pharmaceutical composition for increasing the intestinal motility of an individual, comprising an agonist of CCK-1R in an amount sufficient to increase intestinal motility, and a pharmaceutically acceptable carrier.

An embodiment of the invention encompasses a pharmaceutical composition for decreasing the intestinal motility of an individual, comprising an antagonist of CCK-1R in an amount sufficient to decrease intestinal motility, and a pharmaceutically acceptable carrier.

### BRIEF DESCRIPTION OF THE DRAWINGS

FIG. 1. Small intestinal transit in wild-type (+/+) mice (top panel) and CCK-1R knockout mice (−/−) (bottom panel). Data are determined by the distribution of radioactivity at 30 minutes along the entire length of the small intestine following intraduodenal instillation of [³H]sitostanol dissolved in medium-chain triglyceride. Each bar is the mean percentage of radioactivity in each segment for N=13 mice per group. Segments 1 to 20 represent evenly divided portions from the most proximal to the most distal parts of the small intestine placed on a 50 cm ruler (see Example 1). Arrows indicate the geometric center that is significantly (P<0.001) shorter in CCK-1R (−/−) mice, indicating significantly slower small intestinal transit times (geometric center=7.8±0.8) compared with the wild-type mice (geometric center=10.8±1.0).

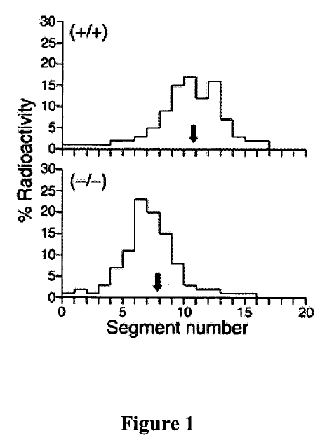

FIG. 2. Percent cholesterol absorption, as determined by the plasma dual isotope ratio method in wild-type (+/+) and CCK-1R (−/−) mice (n=14 per group). The CCK-1R (−/−) mice display significantly (P<0.05) higher intestinal cholesterol absorption efficiencies compared with the wild-type (+/+) mice (see Example 2).

### DETAILED DESCRIPTION OF THE INVENTION

Definitions

As used herein, the term “cholesterol” includes, but is not limited to total serum (in plasma) cholesterol, low-density lipoprotein (LDL), very low-density lipoprotein (VLDL), VLDL+LDL, chylomicron cholesterol, cholesterol esters, and unbound cholesterol.

As used herein, the phrase “reducing the level of cholesterol in an individual” is defined as decreasing the total plasma cholesterol concentration, or decreasing the ratio of plasma low-density lipoprotein concentration to plasma high-density lipoprotein, or decreasing the plasma low-density lipoprotein cholesterol concentration. The desired reduction of plasma cholesterol levels is determined based upon a comparison of the patient's plasma levels with normative values and the physician's professional judgment.

As used herein, the term “administering” applies to any route of administration, including but not limited to oral, rectal, topical, nasal, sub-lingual, and parenteral (e.g. subcutaneous, intramuscular, intradermal, or intravenous). The term “administering” also includes chronic administration which can be applied for example in order to control the patient's cholesterol and triglyceride levels, and/or in order to gain the long-term benefits of atherosclerotic disease treatment and prevention. Such administering includes dosing at regular intervals. The term “administering” also includes acute administration when warranted.

When a compound is used as an agonist or antagonist of CCK-1R in a human subject, the daily dosage will normally be determined by the physician. The dosage generally will vary with the age, weight, and response of the individual patient, as well with as the severity of the patient's symptoms. However, in most instances, a therapeutically effective daily dosage will be in the range of from about 0.001 mg/kg to about 2 mg/kg of body weight; from about 0.01 mg/kg to about 200 ug/kg, e.g., or from about 0.1 mg/kg to about 100 mg/kg of body weight, administered in single or divided doses. One skilled in the art knows that in some cases it will be necessary to use dosages outside these limits. Further guidance for dosages can come from in vitro tests. The active agents of the present methods may be administered in divided doses, for example two or three times daily, or a single daily dose. The active agents, in particular protein agents, may be administered as water soluble salts, generally as salts of alkaline metals such as sodium or potassium salts, as alkylamine salts, preferably diethyl-amine salts or as acid addition salts, preferably the hydrochloride salt.

As used herein, the term “cholecystokinin-1 receptor” or “CCK-1R” refers to a G protein coupled CCK receptor which is expressed in smooth muscle cells of the gall bladder, smooth muscle and neurons within the gastrointestinal tract, and is also found in the brain in discrete regions. The CCK 1 receptor has a much greater affinity (>100 times higher) for CCK than for the related peptide hormone gastrin. The CCK 1 receptor and the CCK 2 receptor are G protein-coupled receptors and share approximately 50% homology. Despite this homology, there are distinct differences between the physiological activity of the CCK 1 receptor and the CCK 2 receptor. The CCK 1 receptor can be distinguished from the CCK 2 receptor, found in the stomach and throughout the CNS, by CCK 2 receptor's property of having a roughly equal ability to bind CCK and gastrin. The amino acid sequences of both these receptors have been determined from cloned cDNA. See U.S. Pat. No. 5,541,071.

As used herein, “CCK” refers to the natural ligand for the CCK 1 receptor. CCK is synthesized as procholecystokinin, a protoprotein of 115 amino acids, (Accession number AAA53094), and is then post-translationally cleaved to a peptide of 33 amino acids, (Accession Number P06307). See U.S. Pat. No. 5,541,071.

As used herein, the term “agonist of cholecystokinin-1 receptor (CCK-1R)” or “CCK-1R agonist” encompasses any compound which binds to the human CCK 1 receptorCCK 2 receptor, and which when bound to the CCK 1 receptor, displays comparable biological activity to endogenous CCK. Thus, a CCK 1 receptor agonist competes with native CCK, and when the CCK 1 receptor agonist is bound to CCK 1 receptor, either causes transduction of the signal which is caused by CCK binding to CCK 1 receptor, either to a greater, lesser or equal degree as that which is transduced by CCK. Accordingly, in some cases a CCK 1 receptor agonist may be a CCK 1 receptor antagonist as defined herein provided that the agonist is a weak agonist. A CCK 1 receptor agonist may also possess antagonist activity with respect to CCK-2R, but in preferred embodiments does not act as a CCK-2R agonist. Representative CCK-1R agonists include but are not limited to GI5269, GI0122, GW5823, GW7854, GW7178, GW8573, a 1,4-benzodiazepine, a 1,5-benzodiazepine, PD170292, SR-146, 131, and a CCK-1R specific agonistic antibody or antigen binding fragment thereof.

As used herein, the term “antagonist of cholecystokinin 1 receptor (CCK-1R)” is defined as a chemical substance that inhibits an activity of the mammalian CCK 1 receptor, such as its ability to bind an agonist. CCK-1R antagonists include but are not limited to, dexioxiglumide, tarazepide, devazepide, lintitript and Loxiglumide, which is the racemate of dexioxiglumide, JMV179, JMV180, SR-27897, L-364718, and a CCK-1R specific antagonistic antidoy or antigen binding fragment thereof.

The term “fragment” can also refer to any CCK polypeptide having an amino acid sequence shorter than that of CCK. As used herein, the term “fragment”, as applied to a polypeptide, will ordinarily be at least about 5 residues, more typically at least about 10, 15, 20, 25, 30, 35, 40, 45, 50, 55 residues, preferably at least about 60 residues in length. Fragments of the CCK can be generated by methods known to those skilled in the art. The ability of a candidate fragment to exhibit a biological activity of a CCK 1 receptor can be assessed by methods known to those skilled in the art as described herein. Also included are CCK polypeptides containing amino acids that are normally removed during protein processing, including additional amino acids that are not required for the biological activity of the polypeptide, or including additional amino acids that result from alternative mRNA splicing or alternative protein processing events.

As used herein, the term “analog” of CCK is defined as a molecule having one or more amino acid substitutions, deletions, inversions, or additions compared with CCK. Analogs can differ from naturally occurring CCK in amino acid sequence, or in modifications that do not affect the sequence, or in both. Analogs of the invention will generally exhibit at least 70%, more preferably 80%, more preferably 90%, and most preferably 95% or even 99%, homology with a naturally occurring CCK sequence.

As used herein, the term “derivative” of CCK is defined as a molecule having the amino acid sequence of CCK or of a CCK analog, but additionally having a chemical modification of one or more of its amino acid side groups, α-carbon atoms, terminal amino group, or terminal carboxylic acid group. Similarly, as used herein, the term “derivative” of CCK-1R is defined as “CCK-1R derivative” is defined as a molecule having the amino acid sequence of CCK-1R or of a CCK-1R analog, but additionally having chemical modification of one or more of its amino acid side groups, α-carbon atoms, terminal amino group, or terminal carboxylic acid group. A chemical modification includes, but is not limited to, adding chemical moieties, creating new bonds, and removing chemical moieties. Modifications at amino acid side groups include, without limitation, acylation of lysine ε-amino groups, N-alkylation of arginine, histidine, or lysine, alkylation of glutamic or aspartic carboxylic acid groups, and deamidation of glutamine or asparagine. Modifications of the terminal amino group include, without limitation, the desamino, N-lower alkyl, N-di-lower alkyl, and N-acyl modifications. Modifications of the terminal carboxyl group include, without limitation, the amide, lower alkyl amide, dialkyl amide, and lower alkyl ester modifications. Lower alkyl is C1-C4 alkyl. Furthermore, one or more side groups, or terminal groups, may be protected by protective groups known to the ordinarily skilled protein chemist. The α-carbon of an amino acid may be mono- or dimethylated. One or more tyrosine residues may be sulfated, such as, for example, the seventh residue from the C-terminus in CCK8. The α-carbon of the C-terminal amino acid may also be amidated.

As used herein, the term “antibody” is defined as an antibody or antigen-binding domain thereof, or a fragment, variant, or derivative thereof, which binds to an epitope on CCK-1R. Antibodies of the invention include polyclonal, monospecific polyclonal, monoclonal, recombinant, chimeric, humanized, fully human, single chain and/or bispecific antibodies, heteroantibodies, or other fragments, variants, or derivatives thereof, which are capable of binding the CCK-1R.

Antibody fragments include those portions of an antibody that bind to an epitope on CCK-1R. Examples of such fragments include but is not limited to Fab, F(ab′), F(ab)′, Fv, fd, dab and sFv fragments. The antibodies may be generated by enzymatic cleavage of full-length antibodies or by recombinant DNA techniques, such as expression of recombinant plasmids containing nucleic acid sequences encoding antibody variable regions. A CCK-1R antibody can have partial or complete agonist or antagonist activity to CCK-1R. Antibody fragments exhibit at least a percentage of the affinity for binding to CCK-1R as the intact antibody from which the fragments were derived, the percentage being in the range of 0.001 percent to 1,000 percent, preferably 0.01 percent to 1,000 percent, more preferably 0.1 percent to 1,000 percent, and most preferably 1.0 percent to 1,000 percent, of the relative affinity of the whole antibody for binding to the CCK-1R.

Monoclonal antibodies may be routinely produced as taught by Harlow, E. and D. Lane, (1988) ANTIBODIES: A Laboratory Manual, Cold Spring Harbor Laboratory, Cold Spring Harbor N.Y., which is incorporated herein by reference. Humanized antibodies may be routinely produced as taught by U.S. Pat. No. 5,585,089 and U.S. Pat. No. 5,530,101, which is incorporated herein by reference. Techniques for engineering antibodies are well known and described in Winter and Millstein (1991) Nature 349:293, and Larrich and Fry (1991) Hum. Antibod. and Hybridomas 2:17, both of which are incorporated herein by reference.

As used herein, the term “high-density lipoprotein” (HDL) refers to a small particle obtaining about 20% cholesterol plus cholesteryl ester, 50% protein, and 25% phospholipids, which appears to play a major role in the exchange of free cholesterol between cells, the liver, and other lipoprotein moieties. Lipoproteins are proteins in the blood that transport cholesterol, triglycerides, and other lipids to various tissues. The main function of HDL appears to be carrying excess cholesterol (and probably other phospholipids and proteins) to the liver for “re-packaging” or excretion in the bile (also known as “reverse cholesterol transport”). Higher levels of HDL seem to be protective against coronary artery disease. Thus, HDL is sometimes referred to as “good” cholesterol. The laboratory test for HDL actually measures the cholesterol part of HDL, not the actual concentration of HDL in the blood. Women tend to have higher HDL cholesterol than men. In general, an increased risk for heart disease, including heart attack, occurs when the HDL level is less than 40mg/dL. More specifically, men are at particular risk if their HDL is below 37 mg/dL, and women, if their HDL is below 47 mg/dL. A HDL 60mg/dL or above helps protect against heart disease. Normal value ranges may vary slightly among different laboratories.

As used herein, the phrase “statistically significant amount” is defined as an index of statistical significance wherein p<0.05, as determined when using statistical analysis including unpaired two-tailed t-test, chi-square analysis, Mann-Whitney U-test, or the correlation coefficient (r), as appropriate, when comparing the change in a parameter (e.g., intestinal motility or serum cholesterol level) for one group of individuals treated similarly (e.g., administered a given dose of a CCK-1R agonist) compared with an untreated (control) group. The significant difference between groups also can be analyzed by one-way analysis of variance, to calculate the p-values by the Dunnett's multiple range test. Statistical significance can also be assessed by measuring the change in a parameter for a single group (i.e., difference before and after treatment) using a paired t test. As used herein, when a parameter is increased or decreased by “a statistically significant amount” in an individual, the amount referred to is that which would be observed in a population of individuals treated in the same manner, and statistical significance is as defined above when comparing the treated population with a controlled population, or when comparing a single population before and after treatment.

As used herein, the phrase “intestinal motility” refers to a series of coordinated, rhythmic propulsive muscle contractions that occur as apart of an automatic and vital process that moves food through the intestine.

As used herein, the phrase “small intestinal motility” refers to a series of coordinated, rhythmic propulsive muscle contractions in the small intestine that moves food through the small intestine.

Intestinal motility can be determined experimentally in the laboratory, for example, by measuring the distribution of radioactivity at 30 minutes along the entire length of the small intestine following intraduodenal instillation of [³H]sitostanol dissolved in medium-chain triglyceride. Alternative non-invasive tests to measure intestinal transit time are based on the use of substrates (e.g., ¹⁴C-xylose) which are either almost exclusively metabolized by bacteria or are malabsorbed and subsequently split by colonic bacteria, e.g., ¹⁴C-glycocholic acid. Isotopically labeled CO₂ or unlabeled metabolites (e.g., H₂) that result from the bacterial degradation of substrates of this type may then be absorbed, transported by the circulation, and finally exhaled by the lungs. Typical tests based on this concept are the ¹³C-urea breath test and the lactulose-hydrogen breath test. Estimates of transit time in the gastrointestinal tract can also be obtained by: (1) the time for initial or peak appearance of hydrogen in breath from the bacterial fermentation of a non-absorbable sugar, for example, lactulose; (2) the appearance of chromium oxide or Styrofoam markers in the stool; or (3) the fluoroscopic examination of the passage of radio-opaque markers through the intestinal tract. Furthermore, transit times of both the small intestine and the colon can be obtained using a pH-sensing radiotelemetry device (see, e.g., Fallingborg et al., Aliment. Pharmacol. Ther. 3:605-13 (1989).

Glycosyl ureides can also be used to measure gastrointestinal motility, and/or to monitor drug transit in the gastrointestinal tract. An oral testing dose of a labeled glycosyl ureide is administered to a subject to be tested, respiratory gas is sampled over time from the subject, and the amount of labeled CO₂ in the respiratory gas is measured. These methods can also be modified to measure the effect of administered CCK-1R agonists or antagonists on gastrointestinal tract motility by first measuring the transit time then administering the treatment, and finally remeasuring the transit time, as described in U.S. Pat. No. 5,233,997. The transit time is measured by administering an oral dose of labeled glycosyl ureide to a subject to be tested, collecting respiratory gas over time from the subject, and measuring the amount of labeled CO₂ in the respiratory gas.

As used herein, the phrase “intestinal motility is increased” means a greater intestinal motility rate relative to a previous motility rate. As used herein, the phrase “small intestinal motility is increased” means a greater small intestinal motility rate relative to a previous intestinal motility rate. Intestinal, or small intestinal motility is increased by a statistically significant amount. Preferably, intestinal or small intestinal motility is increased by at lest about 5%, 10%, 15%, 20%, 25%, 30%, 40%, 50%, or more. In certain embodiments the increase in small intestinal motility is not more than about 25%, so as to avoid diarrhea. In other embodiments, for example to treat constipation, the increase in small intestinal motility can be greater than 25%, up to 50% or more.

As used herein, the phrase “radioopaque tracer” refers to a radiopaque agent which when present at a target site such as the small intestine, allows radiographic viewing. Examples of a radioopaque tracer include, but are not limited to, barium sulfate and meglumine diatrizoate.

As used herein, the phrase “microtelemetry” refers to a method and apparatus for determining intestinal or small intestinal transit time using a biotelemetric device. For example, a pH-sensitive radiotransmitting capsule can be used for this purpose. See, e.g., Fallinbong et al., Aliment. Pharmacol. Ther. 3:605-613 (1989).

As used herein, the phrase “inhibitor of HMG CoA reductase” means any compound that is an inhibitor of cholesterol biosynthesis by virtue of its ability to inhibit the enzyme 3-hydroxy-3-methyl-glutaryl-coenzyme A reductase (HMG-CoA reductase).

As used herein, the phrase “an antihyperlipoproteinemic” refers to any cholesterol lowering drug that comprises a hydrophilic polymer that lowers cholesterol by attracting and binding bile acids in the intestinal tract. Once bound, the bile acids are excreted in feces, and serum LDL levels are lowered. Bile acid sequestrants are known in the art; see, e.g., U.S. Pat. No. 5,451,397.

As used herein, the phrase “an ACAT inhibitor” refers to any inhibitor of acyl-CoA:cholesterol O-acyl transferase (ACAT). As described in U.S. Pat. No. 6,414,002, ACAT inhibitors include Cl-1011 which is effective in the prevention and regression of aortic fatty streak area in hamsters. Nicolosi et al, Atherosclerosis. (1998), 137(1), 77-85; FCE 27677 is an ACAT inhibitor with potent hypolipidemic activity mediated by selective suppression of the hepatic secretion of ApoB100-containing lipoprotein. Ghiselli, Cardiovasc. Drug Rev. (1998), 16(1), 16-30, RP 73163 is a bioavailable alkylsulfinyl-diphenylimidazole ACAT inhibitor, Smith, C., et al, Bioorg. Med. Chem. Lett. (1996), 6(1), 47-50.

As used herein, the phrase “an HMG CoA synthase inhibitor” means a compound which inhibits the biosynthesis of hydroxymethylglutaryl-coenzyme A from acetyl-coenzyme A and acetoacetyl-coenzyme A, catalyzed by the enzyme HMG-CoA synthase. Such inhibition may be determined readily by one of skill in the art according to standard assays (e.g., Methods of Enzymology, 35:155-160 (1975); and Methods of Enzymology, 110: 19-26 (1985); and the references cited therein). A variety of these compounds are described and referenced below. U.S. Pat. No. 5,120,729 discloses certain beta-lactam derivatives. U.S. Pat. No. 5,064,856 discloses certain spiro-lactone derivatives prepared by culturing the microorganism MF5253. U.S. Pat. No. 4,847,271 discloses certain oxetane compounds such as 11-(3-hydroxymethyl-4-oxo-2-oxetayl)-3,5,7-trimethyl-2,4-undecadienoic acid derivatives. Other HMG-CoA synthase inhibitors will be known to those skilled in the art.

As used herein, the phrase “a squalene epoxidase inhibitor” is a nonmetallic flavoprotein monooxygenase that catalyzes the conversion of squalene to 2,3 oxidosqualene, a rate limiting step of cholesterol biosynthesis. Squalene epoxidase is thought to control the throughput of squalenes to sterols in cholesterol biogenesis, and has become a potential target for the design of cholesterol lowering drugs. Squalene epoxidase inhibitors include terbinafine (Lamisil™.) TU-2078 and NB-598, squalene analogs and allylamine derivatives, Abe et al. (2000) Biochem. Biophys. Res. Com. 270:137-140.

As used herein, the term “individual” means any member of an animal species, preferably a vertebrate species, more preferably a mammalian species, and includes a dog, a cat, a horse, a mouse, a rat, a cow, a lamb, a goat, a mouse, a rabbit, a primate, a chimpanzee, a monkey and a human.

As used herein, the disease “hypercholesterolemia” is defined as a condition in an individual characterized by a supranormal total plasma cholesterol, or characterized by a supranormal ratio of plasma low-density lipoprotein cholesterol concentration to plasma high-density lipoprotein cholesterol concentration. In a preferred embodiment, hypercholesterolemia is indicated by a plasma/serum total cholesterol level greater than 140, 150, 160, 170, 180, 190, or 200 mg/dL; or by an HDL cholesterol level of less than 25, 30, 35, 40, 45, or 50 mg/dL; or by an LDL cholesterol level of greater than 90, 100, 110, 120, or 130 mg/dL; or by a total cholesterol: HDL cholesterol ratio of more than 5.1 or an LDL cholesterol: HDL cholesterol ratio of more than 3.7.

As used herein, the disease “atherosclerosis” is defined as a condition in which fatty material is deposited along the walls of arteries. This fatty material thickens, hardens, and may eventually block the arteries. The hardening of the arteries which is characterized by thickening and hardening of artery walls

As used herein, the disease “myocardial infarction” is defined as a heart attack which occurs when an area of heart muscle dies or is permanently damaged because of an inadequate supply of oxygen to that area. Most heart attacks are caused by a clot that blocks one of the coronary arteries. The clot usually forms in a coronary artery that has been previously narrowed from changes related to atherosclerosis. The atherosclerotic plaque (buildup) inside the arterial wall sometimes cracks, and this triggers the formation of a clot, also called a thrombus.

A clot in the coronary artery interrupts the flow of blood and oxygen to the heart muscle, leading to the death of heart cells in that area. The damaged heart muscle loses its ability to contract, and the remaining heart muscle needs to compensate for the damaged heart muscle.

As used herein, the disease “stroke” is defined as an interruption of the blood supply to any part of the brain, resulting in damaged brain tissue. A stroke can also be a hemorragic stroke, in which a blood vessel in the brain ruptures and results in damage to brain tissue.

As used herein, the disease “gallstones” is defined as a precipitation of the components of bile, principally cholesterol, forming gallstones. Gallstones are formed within the gallbladder, an organ that stores bile excreted from the liver. Bile is a solution in water, of bile salts, phospholipids, cholesterol, and other substances. If the concentration of these components changes, cholesterol may precipitate from solution and form gallstones.

As used herein, the disease “Alzheimer's disease” is defined as a form of dementia, and is a progressive, degenerative brain disease. It impairs memory, thinking, and behavior. The term “Alzheimer's disease” includes early onset and late onset Alzheimer's disease. In Alzheimer's disease, the brain tissue shows “neurofibrillary tangles” (twisted fragments of protein within nerve cells that clog up the cell), “neuritic plaques” (abnormal clusters of dead and dying nerve cells, other brain cells, and protein), and “senile plaques” (areas where products of dying nerve cells have accumulated around protein).

As used herein, the disease “constipation” is defined as infrequent or hard stools, or difficulty passing stools. Constipation may involve pain during the passage of a bowel movement, inability to pass a bowel movement after straining or pushing for more than 10 minutes, or no bowel movements after more than 3 days.

As used herein, the disease “gastric stasis,” is defined as a condition in which the stomach's ability to empty its contents is impaired, unrelated to obstruction.

As used herein, the disease “irritable bowel syndrome” is defined as a disorder that interferes with the normal functions of the large intestine (colon), characterized by a group of symptoms including crampy abdominal pain, bloating, constipation, and diarrhea.

As used herein, the disease “inflammatory bowel disease” is defined as a chronic inflammation of the intestines which is usually confined to the terminal portion of the small intestine, the ileum.

As used herein, the phrase “treating a condition” is defined as administering pharmaceuticals and/or applying medical procedures to an individual, who has said condition, a symptom of said condition, or a predisposition toward said condition, with the purpose to cure, heal, alleviate, relieve, alter, remedy, ameliorate, improve, or affect said condition, the symptoms of said condition, or the predisposition toward said condition. The terms “treating”, “treat” or “treatment” include preventative (e.g., prophylactic) and palliative treatment. In a preferred embodiment, the treatment comprises administering an agonist or antagonist of a CCK-1R, alone or in combination with other drugs or nutrients and/or treatment regimes, such as hyperalimentation therapy.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician.

Description of the Embodiments

The inventors have discovered that hypercholesterolemia can be treated using methods that increase intestinal motility, preferably small intestinal motility, by increasing the activity of the CCK 1 receptor. A decreased transit time of cholesterol-containing material in the intestine, particularly in the small intestine, mediated by increased intestinal motility caused by activation of the CCK 1 receptor, results in a decreased amount of cholesterol being absorbed by the intestines, in particular by the small intestine. The CCK 1 receptor activity can be increased by many methods, including but limited to, through the use of CCK-1R agonists.

The present inventors have also discovered that the amount of intestinal absorption of drug or nutrient materials, including but not limited to hydrophobic drugs or nutrients, can be increased using methods that decrease intestinal motility, preferably small intestinal motility, by decreasing the activity of the CCK 1 receptor. An increased transit time of a drug in the intestine, particularly in the small intestine, mediated by decreased intestinal motility caused by inhibition of the CCK 1 receptor, results in an increased amount of drug or nutrient being absorbed by the intestines, in particular by the small intestine. The CCK 1 receptor activity can be decreased by many methods, including but limited to, through the use of CCK-1R antagonists.

This invention encompasses a method of reducing the level of cholesterol in an individual, comprising administering to said individual an agonist of a cholecystokinin-1 receptor (CCK-1R), whereby the level of cholesterol is reduced in said individual. The encompassed methods of reducing cholesterol can be applied to any warm-blooded animal, including, but not limited to, a mammal. The mammal includes but is not limited to a dog, a cat, a horse, a mouse, a rat, a cow, a lamb, a goat, a mouse, a rabbit, a primate, a chimpanzee, a monkey and a human. The characteristics of patients at risk of having atherosclerosis are well known to those in the art and include patients who have a family history of cardiovascular disease, including hypertension and atherosclerosis, obese patients, patients who exercise infrequently, patients with hypercholesterolemia, hyperlipidemia and/or hypertriglyceridemia, patients having high levels of LDL or Lp(a), patients having low levels of HDL (hypoalphalipoproteinemia), and the like.

A CCK 1 receptor encompassed by the invention includes but is not limited to a receptor which originates from the above listed animals, most preferably a human. One or more CCK 1 receptor agonists can be administered to reducing the level of cholesterol in an individual. The administered agonist can include, but is not limited to the following; CCK or a fragment, analog or derivative thereof; GI5269; GI0122; GW5823; GW7854; GW7178; GW8573; a 1,4-benzodiazepine; a 1,5-benzodiazepine; PD170292; SR-146,131; and a CCK-1R specific agonistic antibody or antigen binding fragment thereof. Further, the agonist can be administered in conjunction with other agents, including but not limited to cholesterol reducing agents.

The type of cholesterol reduced can be any form of cholesterol in the mammalian body, either alone or in combination with other forms of cholesterol in the mammalian body. For example, the type of cholesterol can be selected from the group consisting of total serum (or plasma) cholesterol, low-density lipoprotein (LDL), very low-density lipoprotein (VLDL), VLDL+LDL chylomicron cholesterol, cholesterol esters, and unbound cholesterol. In another aspect of this method, the administration of the CCK-1R agonist reduces the cholesterol level by a statistically significant amount. In some embodiments, the statistically significant amount of reduction in cholesterol level is at least 1%, 2%, 3%, 4%, 5%, 6%, 7%, 8%, 9%, 10%, 15%, 20%, 25% or more. In another aspect of this method, the statistically significant amount of reduction in cholesterol level is relative to the cholesterol in said individual before the administration to said individual an agonist of a cholecystokinin-1 receptor.

This invention encompasses a method of reducing the level of cholesterol in an individual, comprising administering to said individual an agonist of a cholecystokinin-1 receptor (CCK-1R), whereby the high-density lipoprotein (HDL) is increased in said individual.

This invention encompasses a method of reducing the level of cholesterol in an individual, comprising administering to said individual an agonist of a cholecystokinin-1 receptor (CCK-1R), and further comprising measuring the level of cholesterol in said individual either prior to administering said agonist or after administering said agonist. In some embodiments, the level of cholesterol is measured both before and after the administration of said CCK-1R agonist. In yet other embodiments, the dose of CCK-1R agonist is adjusted following determination of the level of cholesterol in said individual.

This invention encompasses a method of reducing the level of cholesterol in an individual, wherein the intestinal motility is increased in said individual. In an aspect of this method, the small intestinal motility is increased in said individual. In a further aspect of this method, the intestinal motility is measured. The intestinal motility in said individual can be measured either prior to administering said agonist or after administering said agonist. The intestinal motility can be measured by any known means, for example, by using a radioopaque tracer or microtelemetry.

This invention encompasses a method of reducing the level of cholesterol in an individual, comprising administering to said individual an agonist of a cholecystokinin-1 receptor (CCK-1R), and further comprising administering an additional cholesterol reducing agent to said individual. This cholesterol reducing agent can include, but is not limited to the following: a lipase inhibitor, tetrahydrolipstatin (THL), [orlistat (lipstatin, Roche)], a bile acid sequestrant such as cholestyramine, colestipol and DEAE-Sephadex (Secholexg, Polidexide®), as well as cholesevelan HCl, lipostabil (Rhone-Poulenc), Eisai E-5050 (an N-substituted ethanolamine derivative), imanixil (HOE-402), istigmastanylphosphorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, neomycin, p-aminosalicylic acid, aspirin, poly-(diallylmethylamine) derivatives such as disclosed in U.S. Pat. No. 4,759,923, quaternary amine poly(diallyldimethylammonium chloride) and ionenes such as disclosed in U.S. Pat. No. 4,027,009, lovastatin, parvastatin, simvastatin, probucol, squalene, gemfibrozil, endomycin, niacin, an inhibitor of HMG CoA reductase, synvolin, pravastain, an antihyperlipoproteinemic, an ACAT inhibitor, an HMG CoA synthase inhibitor, an HMG CoA reductase inhibitor, a squalene epoxidase inhibitor, and other known serum cholesterol lowering agents. In another aspect of this method of reducing the level of cholesterol in an individual, the route of administration includes but is not limited to, the oral, nasal, or parenteral administration of an agonist of a cholecystokinin-1 receptor (CCK-1R).

This invention presents methods of modulating intestinal motility through the use of CCK-1R agonists and antagonists. Methods comprising the administration of CCK-1R agonists are aimed at increasing intestinal motility rates, which in turn results in decreased small intestine transit times. Decreased transit times result in decreased absorption rates of intestinal material, especially hydrophobic material, such as cholesterol. Accordingly, administration of CCK-1R agonists results in a decrease in the cholesterol absorption by the small intestine, which produces lower levels of serum cholesterol.

Structural models provide detailed guidance to the person of ordinary skill in the art as to the construction of a variety of binding elements able to retain the binding characteristics of biologically active CCK peptides for the CCK 1 receptor. Studies of the interaction between the CCK 1 receptor and CCK have shown that the primary receptor sequence region containing amino acid residues 38 through 42 is involved in the binding of CCK. These residues do not appear to be essential for the binding of CCK analogs JMV 180 (corresponding the synthetic C-terminal heptapeptide of CCK in which the phenylalanylamide residue is substituted by a phenylethyl ester and the threonine is substituted with norleucine), and JMV 179 (in which the phenylalanylamide residue and the L-tryptophan residues of the synthetic CCK nonapeptide are substituted by a phenylethyl ester and D-tryptophan, respectively and the threonine is substituted with norleucine). These studies by Kennedy et al., J. Biol. Chem. 272: 2920-2926 (1997), and similar studies have shed light on the structure of the CCK 1 receptor active site. Based on receptor binding experiments, a current structural model indicates that CCK residues Trp₃₀ and Met₃₁ (located at positions 4 and 3, respectively, from the C terminus of mature CCK-8) reside in a hydrophobic pocket formed by receptor residues Leu₃₄₈, Pro₃₅₂, Ile₃₅₃ and Ile₃₅₆. CCK residue Asp₃₂ (located at amino acid position 2 measured from the C terminus of CCK-8) seems to be involved in an ionic interaction with receptor residue Lys₁₁₅. CCK Tyr-sulfate₂₇ (the CCK-8 residue 7 amino acids from C terminus) appears involved in an ionic interaction with receptor residue Lys₁₁₅ and a stacking interaction with receptor residue Phe₁₉₈. Ji, et al., 272 J. Biol. Chem. 24393-24401 (1997).

Conversely, methods comprising the administration of CCK-1R antagonists are aimed at decreasing intestinal motility rates, which in turn results in an increased transit time in the small intestine. An increase in transit time results in an increased absorption rate of intestinal material, especially hydrophobic material or material encompassed by a hydrophobic carrier. Accordingly, administration of CCK-1R antagonists results in an increase in the absorption of drugs and other material such as nutrients, especially hydrophobic material, by the small intestine, including material coupled to a hydrophobic carrier. Therefore, this invention encompasses methods of increasing the absorption of one or more drugs or nutrients of interest by administering an antagonist of CCK-1R. Drugs of interest include estrogens, progestogens, ursodiol, antivirals for HIV and Herpes Simplex (see, e.g., PDR), immunosuppressives (see, e.g., PDR), antilipoproteinemic drugs, cholesterol lowering agents, prostaglandins, and antibiotics. Nutrients include fats, oils, lipids, amino acids, proteins, vitamins, sugars, carbohydrates, raw, cooked, or partially digested foodstuffs, and mixtures of natural or artificial substances for nutrition. Hydrophobic carriers of interest include Eudragit, microvesicles, hydrophobic silicone spheres, etc., and any drug administered using a lipid phase delivery system (e.g., any liposome-encapsulated or micelle-embedded drug).

An assay may be used to test compounds to determine whether or not they are CCK 1 receptor specific ligands, or whether they possess CCK 1 receptor binding activity. Compositions that specifically bind to CCK 1 receptors can be identified by a competitive binding assay. The competitive binding assay is a standard technique in pharmacology, which can be readily performed by those having ordinary skill in the art using readily available starting materials. Competitive binding assays, have been shown to be effective for identifying compositions that specifically bind to receptors. To identify CCK 1 receptor specific ligands, or a second assay is performed using the CCK 2 receptor and the results are compared.

Briefly, an assay to identify CCK receptor ligands consists of incubating a preparation of CCK 1 receptors with a constant concentration (e.g. 1×10⁻¹⁰M to 5×10⁻¹⁰M) of labeled CCK and a known concentration, or a range of concentrations, of a test compound. As a control, a duplicate preparation of CCK 1 receptors is incubated with a duplicate concentration of labeled CCK in the absence of test compound. Assays are incubated to equilibrium (2 hours) and the amount of CCK bound to receptors is quantified by standard techniques. The ability of the test compound to bind to receptors is measured as its ability to prevent (compete with) the labeled CCK from binding. Thus, in assays containing a test compound which binds to the receptor, there will be less label associated with the receptors. This assay, which is appropriate for determining the ability of any molecule to bind to CCK 1 receptors, is a standard competitive binding assay which can be readily employed by those having ordinary skill in the art using readily available starting materials. A parallel assay may be run using CCK 2 receptors instead of CCK 1 receptors, and screening for those ligands which preferentially bind to CCK 1 receptors. Ligands which preferentially bind CCK 1 receptors are CCK 1 receptor specific ligands.

Alternatively, CCK 1 receptor ligands can be identified using a serum response element (SRE) reporter gene construct, for example an SRE-luciferase construct. Briefly, cells bearing CCK 1 receptor (or transfected so as to express CCK 1 receptor) are transfected with a multimerized SRE cloned upstream from the coding sequence for firefly luciferase. The cells are transfected using LipofectAMINE (Invitrogen) and stimulated for 24 hours at 37° C. in serum-free medium (e.g., DMEM (Gibco/BRL)). The cells are then incubated an additional 18 hours with either CCK-8 (3×10⁻⁷ M), a candidate ligand, or no ligand (control for basal activity). The cells are then lysed and assayed for light emission, for example using a LucLite luciferase assay kit (Packard). See Kopin et al., PNAS 100:5525-30 (2003).

Yet another alternative to determine CCK-1R activation and screen for ligands of CCK-1R is to measure inositol phosphate production. For example, cells can be labeled overnight with 3 microCi/ml myo-[³H]inositol in serum-free DMEM. Cells are then stimulated for 1 hour at 37° C. with either CCK-8 (3×10⁻⁷ M), a candidate ligand, or no ligand (control for basal activity) in PBS (Gibco/BRL) containing 10 mM LiCl. After stimulation, the cells are lysed and extracted with methanol/chloroform. The upper phase is analyzed for IPs using strong anion exchange chromatography as described by Beinborn et al., J. Biol. Chem. 273:14146-51 (1998).

### EXAMPLES

### Example 1

Knockout mice were prepared to evaluate the role of CCK-1R in cholesterol absorption. CCK-1R (−/−) mice displayed significantly slower small-intestinal transit times compared with wild-type mice as indicated by the distributions of radioactivity administered as [³H]sitostanol along the length of the small intestines of CCK-1R (−/−) and wild-type mice.

In detail, male homozygous CCK-1R knockout mice and wild-type mice of the same 129/SvEv background (Charles River Laboratories, Wilmington, Mass., USA) at 3-6 months of age were used in the following examples. All animals were maintained in a temperature-controlled room (22±1° C.) with 12-hour light (6 am-6 pm) cycles. Mice were allowed free access to water and standard Purina rodent chow (Purina Mills Inc., St. Louis, Mo., USA), which contains trace quantities (<0.02%) of cholesterol. After being fed chow for 14 days, small intestine transit times were measured in each group of mice. Under pentobarbital anesthesia, 2 μCi of [³H]sitostanol—as a nonabsorbable reference marker, dissolved in 100 μl of medium-chain triglyceride—was instilled into the small intestine of mice via a previously fitted in situ externalized duodenal catheter. Exactly 30 minutes after instillation, mice were again anesthetized with an intraperitoneal injection of 35 mg/kg pentobarbital. The abdomen was opened, and stomach, small and large intestines, and cecum were removed rapidly while avoiding digital or instrumental compression. The small intestine was frozen promptly in liquid N₂, placed on a 50-cm ruled template, and cut into 20 equal segments with a scalpel blade. Individual segments were placed in tubes containing 10 ml of CHCl₃—CH₃OH (2:1, vol/vol), homogenized, and centrifuged at 10,000×g for 30 minutes. The samples were then stored at 4° C. for 48 hours. Well-mixed portions (1 ml) were pipetted into counting vials, and the solvent was evaporated under N₂. EcoLite (7 ml) was then added, and radioactivity was determined by liquid scintillation counting. Samples of stomach, cecum, and large intestine were also analyzed, but none showed appreciable radioactivity above background. Using these data, a calculation of small-intestinal transit time was carried out by two arithmetic methods: (a) the percentages of total [³H]sitostanol radioactivity in each of the 20 small-intestinal segments were transformed to cumulative percentages passing each segment; (b) the geometric center for the distribution of radioactivity within the small intestine was derived from the sum of the proportions of [³H]sitostanol per segment multiplied by segment number.

Distributions of radioactivity lengthwise throughout the small intestine were significantly (P<0.01) different between the wild-type and CCK-1R (−/−) mice, with peaks occurring between segments 8 and 15 in wild-type mice compared with segments 4 and 11 in CCK-1R (−/−) mice. The geometric center of the [³H]sitostanol distribution profiles in the small intestine of wild-type mice was 10.8±1.0 whereas in CCK-1R (−/−) mice the value was 7.8±0.8. This significant (P<0.001) difference, was consistent with slower small intestinal transit times in CCK-1R (−/−) mice.

These results indicate that a physiologically relevant mechanism mediated by CCK-1R regulates small-intestinal motility appreciably. Furthermore, as illustrated in the next example, retardation of small-intestinal transit time enhances cholesterol absorption from the intestine.

### Example 2

This example shows that CCK-1R−/− mice displayed significantly (P<0.01) higher intestinal cholesterol absorption efficiencies when compared to wild-type mice. The intestinal cholesterol absorption was measured by the dual isotope method. Mice were anesthetized lightly by intraperitoneal injection of pentobarbital (35 mg/kg). An incision of 0.4 cm was made on the neck, and the jugular vein was exposed. Exactly 2.5 μCi of [³H]cholesterol in 100 μl of Intralipid was injected with a 100 μl Hamilton syringe fitted with a 30-gauge needle. The incision was closed with 3-0 silk sutures. A feeding needle with round tip (18 gauge, 50 mm in length) was then inserted into the stomach of the mouse, and each animal was given an intragastric bolus of 1 μCi of [¹⁴C]cholesterol in 150 μl of medium-chain triglyceride oil by gavage. After dosing, mice were returned to individual cages with wire mesh bottoms, where they were free to eat chow for an additional 3 days. Animals were then anesthetized, and were bled from the heart into heparinized microtubes. Plasma was obtained by centrifugation at 10,000 g for 30 min at room temperature. To determine the proportions of [¹⁴C]cholesterol and [³H]cholesterol doses remaining in plasma at 3 days, 10 ml of EcoLite (ICN Biomedicals, Costa Mesa, Calif.) was added to 100-μl portions of plasma and the original dosing mixture, respectively. The vials were shaken vigorously for 10 min, and counted in a liquid scintillation spectrometer.

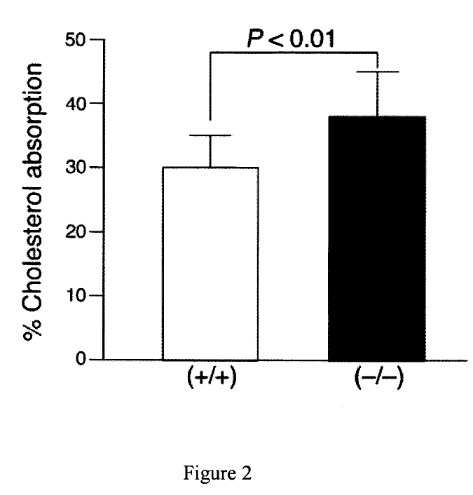

The radioisotope ratio in plasma was used for calculating the percentage cholesterol absorption as follows: Percentage cholesterol absorption=(percentage of intragastric dose of [¹⁴C]cholesterol per milliliter plasma/percentage of i.v. dose of [³H]cholesterol per milliliter plasma). Displayed in FIG. 2 are values for percent cholesterol absorption as calculated from the plasma ratios of [¹⁴C] and [³H]cholesterol 3d after dosing. Percent cholesterol absorption values are significantly (P<0.01) larger in CCK-1R (−/−) mice (38±7%) compared with wild-type mice (30±5%). Since chow contains trace cholesterol (<0.02%), and as both CCK-1R (−/−) and wild-type mice ate similar amounts of food (4.3-4.5 g/day), the total cholesterol mass absorbed from the small intestine was calculated to be 0.33 mg/d in CCK-1R (−/−) mice and ˜0.26 mg/d in wild-type mice.

Though not being limited or held to any one mechanism, these results indicate that a physiologically relevant mechanism mediated by CCK-1R regulates small-intestinal motility appreciably. Furthermore, while not adhering to this mechanism or any mechanism or mode of action, a retardation of small-intestinal transit time enhances cholesterol absorption from the intestine, most likely because a longer residence time of the sterol in the small-intestinal lumen. This, in turn, would increase cholesterol's incorporation into mixed micelles and also would promote partitioning of cholesterol monomers out of micelles, rendering them available for intestinal capture by cholesterol influx transporter(s) on apical membranes of small-intestinal enterocytes.

### Example 3

Examples 1 and 2 demonstrate two important aspects of the instant invention: (a) small-intestinal motility is, in part, mediated by CCK-1R-induced signaling, and (b) the CCK-1R control of small-intestinal transit times is a physiological response that is an important influence in modulating intestinal cholesterol absorption.

This example shows that the administration of an agonist of CCK-1R increases intestinal motility, decreasing the small-intestinal transit time, decreasing cholesterol absorption, resulting in a reduced serum cholesterol level relative to that before the administration of the agonist. Wild-type mice (129/SvEv Charles River Laboratories, Wilmington, Mass., USA) at 3-6 months of age are divided into two groups. The first group, which is the test group, is administered a CCK-1R agonist in a pharmaceutical carrier. The second group, which is the control group, is administered the pharmaceutical carrier without the agonist. The control mice display significantly higher intestinal cholesterol absorption efficiencies compared with test mice when the intestinal cholesterol absorption is measured by the dual isotope method described above in Example 2.

The radioisotope ratio in plasma is used for calculating the percentage cholesterol absorption as follows: Percentage cholesterol absorption=(percentage of intragastric dose of [¹⁴C]cholesterol per milliliter plasma/percentage of i.v. dose of [³H]cholesterol per milliliter plasma). Percent cholesterol absorption values are significantly larger in control mice compared with test mice.

### Example 4

In this example mice are treated with an antagonist of CCK-1R and show decreased small intestinal motility relative to mice who are not treated with the antagonist. Wild-type mice (129/SvEv Charles River Laboratories, Wilmington, Mass., USA) at 3-6 months of age are divided into two groups. The first group, which is the test group, is administered a CCK-1R antagonist in a pharmaceutical carrier. The second group, which is the control group, is administered the pharmaceutical carrier without the antagonist. The test mice display significantly slower small-intestinal transit times compared with the control mice as indicated by the distributions of radioactivity administered as [³H]sitostanol along the length of the small intestines of test and control mice. These data are obtained at exactly 30 minutes following intraduodenal instillation of the radioisotope dissolved in medium chain triglyceride, as described in Example 1.

The distributions of radioactivity lengthwise throughout the small intestine are significantly different between the test and control mice. The geometric center of the [³H]sitostanol distribution profiles in the small intestine of control mice is greater than that in the test mice, consistent with slower small intestinal transit times in the test mice, who are administered the antagonist of CCK-1R.

Variations, modifications, and other implementations of what is described herein will occur to those of ordinary skill in the art without departing from the spirit and scope of the invention. All patents, patent applications, and published references cited herein are hereby incorporated by reference in their entirety.

While this invention has been particularly shown and described with references to preferred embodiments thereof, it will be understood by those skilled in the art that various changes in form and details may be made therein without departing from the scope of the invention encompassed by the appended claims. Those skilled in the art will recognize that other embodiments and configurations known in the art would be within the spirit and scope of the present invention.

1. A method of reducing the level of cholesterol in an individual, comprising administering to said individual an agonist of a cholecystokinin-1 receptor (CCK-1R), whereby the level of cholesterol is reduced in said individual.

2. The method of claim 1, wherein said agonist is selected from the group consisting of CCK or a fragment, analog or derivative thereof, GI5269, GI0122, GW5823, GW7854, GW7178, GW8573, a 1,4-benzodiazepine, a 1,5-benzodiazepine, PD170292, SR-146,131, and a CCK-1R specific agonistic antibody or antigen binding fragment thereof.

3. The method of claim 1, wherein the type of cholesterol reduced is selected from the group consisting of total serum cholesterol, low-density lipoprotein (LDL), very low-density lipoprotein (VLDL), VLDL+LDL and chylomicron cholesterol, cholesterol esters, and unbound cholesterol.

4. The method of claim 1, wherein high-density lipoprotein (HDL) is increased in said individual.

5. The method of claim 1, wherein said administration reduces the cholesterol level by a statistically significant amount.

6. The method of claim 1, wherein said administration reduces the cholesterol level by at least 5%.

7. The method of claim 1, further comprising measuring the level of cholesterol in said individual either prior to administering said agonist or after administering said agonist.

8. The method of claim 7, wherein the type of cholesterol measured is selected from the group consisting of total serum cholesterol, low-density lipoprotein (LDL), very low-density lipoprotein (VLDL), VLDL+LDL chylomicron cholesterol, cholesterol esters, and unbound cholesterol.

9. The method of claim 1, wherein intestinal motility is increased in said individual.

10. The method of claim 9, wherein small intestinal motility is increased.

11. The method of claim 1, further comprising measuring the intestinal motility in said individual either prior to administering said agonist or after administering said agonist.

12. The method of claim 11, wherein the intestinal motility is measured by using a radioopaque tracer or microtelemetry.

13. The method of claim 1, further comprising administering an additional cholesterol reducing agent to said individual.

14. The method of claim 13, wherein said cholesterol reducing agent is selected from the group consisting of a lipase inhibitor, cholestyramine, cholestipol, a bile acid sequestrant, lovastatin, parvastatin, simvastatin, probucol, gemfibrozil, endomycin, niacin, an inhibitor of HMG CoA reductase, synvolin, pravastain, an antihyperlipoproteinemic, an ACAT inhibitor, an HMG CoA synthase inhibitor, and a squalene epoxidase inhibitor.

15. The method of claim 1, wherein said administration is oral, nasal, or parenteral.

16. The method of claim 1, wherein said individual is a human.

17. The method of claim 1, wherein said CCK-1R is a human CCK-1R.

18. A method of treating a condition selected from the group consisting of hypercholesterolemia, atherosclerosis, myocardial infarction, stroke, gallstones, Alzheimer's disease, constipation, gastric stasis, irritable bowel syndrome, and inflammatory bowel disease; said method comprising administering to an individual in need thereof an agonist of a CCK-1R in a therapeutically effective amount, wherein intestinal motility is increased in said individual.

19. The method of claim 18, wherein said agonist is selected from the group consisting of CCK or a fragment, analog or derivative thereof, GI5269, GI0122, GW5823, GW7854, GW7178, GW8573, a 1,4-benzodiazepine, a 1,5-benzodiazepine, PD170292, SR-146,131, and a CCK-1R specific agonistic antibody or antigen binding fragment thereof.

20. The method of claim 18, wherein intestinal motility is increased in said individual.

21. The method of claim 20, wherein small intestinal motility is increased.

22. The method of claim 18, further comprising measuring the intestinal motility in said individual either prior to administering said agonist or after administering said agonist.

23. The method of claim 22, wherein the intestinal motility is measured by using a radioopaque tracer or microtelemetry.

24. The method of claim 18, wherein said administration is oral, nasal, or parenteral.

25. The method of claim 18, wherein said individual is a human.

26. The method of claim 18, wherein said CCK-1R is a human CCK-1R.

27. The method of claim 18, further comprising administering a drug useful to treat said condition.

28. A method of increasing the intestinal absorption of a drug or nutrient, comprising administering to an individual a composition comprising an antagonist of a CCK-1R in an amount sufficient to decrease intestinal motility in said individual, whereby the intestinal absorption of said drug or nutrient is increased in said individual.

29. The method of claim 28, wherein said drug is hydrophobic.

30. The method of claim 28, wherein said nutrient is a dietary supplement or hyperalimentation supplement.

31. The method of claim 28, wherein said drug or nutrient is selected from the group consisting of an estrogen, a progestogen, ursodiol, an antiviral for HIV or Herpes Simplex, an immunosuppressive, an antilipoproteinemic drug, a cholesterol lowering agent, a prostaglandin, an antibiotic, a fat, an oil, a lipid, an amino acid, a protein, a vitamin, a sugar, a carbohydrate, a foodstuff, a mixture of natural or artificial substances for nutrition, a hydrophobic carrier, Eudragit, microvesicles, hydrophobic silicone spheres, and a drug administered using a lipid phase delivery system.

32. The method of claim 29, wherein said hydrophobic drug has an octanol/water partition coefficient in the range from about 0.01 to about 25.

33. The method of claim 28, wherein said antagonist is selected from the group consisting of tarazepide, devazepide, lintitript, dexioxiglumide, loxiglumide, JMV179, JMV 180, SR-27,897, L-364,718, and a CCK-1R specific antagonistic antibody or antigen binding fragment thereof.

34. The method of claim 28, wherein the intestinal absorption of said drug or nutrient is increased by a statistically significant amount.

35. The method of claim 28, wherein the intestinal absorption of said drug or nutrient is increased by at least 5%.

36. The method of claim 28, wherein the small intestinal motility of said individual is decreased.

37. The method of claim 28, wherein said antagonist is administered orally, nasally, or parenterally.

38. The method of claim 28, wherein said individual is a human.

39. The method of claim 28, wherein said CCK-1R is a human CCK-1R.

40. A method of increasing intestinal motility in an individual, comprising administering to said individual an agonist of CCK-1R in an amount sufficient to increase the intestinal motility of said individual.

41. The method of claim 40, wherein said agonist is selected from the group consisting of GI5269, GI0122, GW5823, GW7854, GW7178, GW8573, a 1,4-benzodiazepine, a 1,5-benzodiazepine, PD170292, SR-146,131, CCK, or fragment, analog or derivative thereof, and a CCK-1R specific agonist antibody or antigen binding fragment thereof.

42. The method of claim 40, wherein small intestinal motility is increased.

43. The method of claim 40, wherein said administration is oral, nasal, or parenteral.

44. The method of claim 40, wherein said individual is a human.

45. The method of claim 40, wherein said CCK-1R is a human CCK-1R.

46. A method of decreasing intestinal motility in an individual, comprising administering to said individual an antagonist of CCK-1R in an amount sufficient to decrease intestinal motility of said individual.

47. The method of claim 46, wherein said antagonist is selected from the group consisting of tarazepide, devazepide, lintitript, dexioxiglumide, loxiglumide, JMV179, JMV 180, SR-27,897, L-364,718, and a CCK-1R specific antagonist antibody and antigen binding fragment thereof.

48. The method of claim 46, wherein small intestinal motility is decreased.

49. The method of claim 46, wherein said administration is oral, nasal, or parenteral.

50. The method of claim 46, wherein said individual is a human.

51. The method of claim 46, wherein said CCK-1R is human CCK-1R.

52. A pharmaceutical composition for reducing the cholesterol level of an individual, comprising an agonist of CCK-1R in an amount sufficient to decrease intestinal absorption of cholesterol in said individual, and a pharmaceutically acceptable carrier.

53. The pharmaceutical composition of claim 52, further comprising an effective amount of a cholesterol reducing agent.

54. A pharmaceutical composition for increasing the intestinal absorption of a therapeutic drug or nutrient in an individual, comprising an antagonist of CCK-1R in an amount sufficient to increase intestinal absorption of said therapeutic drug or nutrient, and a pharmaceutically acceptable carrier.

55. The pharmaceutical composition of claim 54, further comprising a therapeutically effective amount of said therapeutic drug or nutrient.

56. A pharmaceutical composition for increasing the intestinal motility of an individual, comprising an agonist of CCK-1R in an amount sufficient to increase intestinal motility, and a pharmaceutically acceptable carrier.

57. A pharmaceutical composition for decreasing the intestinal motility of an individual, comprising an antagonist of CCK-1R in an amount sufficient to decrease intestinal motility, and a pharmaceutically acceptable carrier.


SEQUENCE LISTING (4 sequences)

SEQ ID NO: 1 - protein, 115 aa, Homo sapiens
MNSGVCLCVLMAVLAAGALTQPVPPADPAGSGLQRAEEAPRRQLRVSQRTDGESRAHLGALLARYIQQARKAPSGRMSIVKNLQNLDPSHRISDRDYMGWMDFGRRSAEEYEYPS

SEQ ID NO: 2 - protein, 33 aa, Homo sapiens
KAPSGRMSIVKNLQNLDPSHRISDRDYMGWMDF

SEQ ID NO: 3 - protein, 428 aa, Homo sapiens
MDVVDSLLVNGSNITPPCELGLENETLFCLDQPRPSKEWQPAVQILLYSLIFLLSVLGNTLVITVLIRNKRMRTVTNIFLLSLAVSDLMLCLFCMPFNLIPNLLKDFIFGSAVCKTTTYFMGTSVSVSTFNLVAISLERYGAICKPLQSRVWQTKSHALKVIAATWCLSFTIMTPYPIYSNLVPFTKNNNQTANMCRFLLPNDVMQQSWHTFLLLILFLIPGIVMMVAYGLISLELYQGIKFEASQKKSAKERKPSTTSSGKYEDSDGCYLQKTRPPRKLELRQLSTGSSSRANRIRSNSSAANLMAKKRVIRMLIVIVVLFFLCWMPIFSANAWRAYDTASAERRLSGTPISFILLLSYTSSCVNPIIYCFMNKRFRLGFMATFPCCPNPGPPGARGEVGEEEEGGTTGASLSRFSYSHMSASVPPQ

SEQ ID NO: 4 - protein, 447 aa, Homo sapiens
MELLKLNRSVQGTGPGPGASLCRPGAPLLNSSSVGNLSCEPPRIRGAGTRELELAIRITLYAVIFLMSVGGNMLIIVVLGLSRRLRTVTNAFLLSLAVSDLLLAVACMPFTLLPNLMGTFIFGTVICKAVSYLMGVSVSVSTLSLVAIALERYSAICRPLQARVWQTRSHAARVIVATWLLSGLLMVPYPVYTVVQPVGPRVLQCVHRWPSARVRQTWSVLLLLLLFFIPGVVMAVAYGLISRELYLGLRFDGDSDSDSQSRVRNQGGLPGAVHQNGRCRPETGAVGEDSDGCYVQLPRSRPALELTALTAPGPGSGSRPTQAKLLAKKRVVRMLLVIVVLFFLCWLPVYSANTWRAFDGPGAHRALSGAPISFIHLLSYASACVNPLVYCFMHRRFRQACLETCARCCPRPPRARPRALPDEDPPTPSIASLSRLSYTTISTLGPG